SEQUENCE LISTING (12 sequences)

SEQ ID NO: 1 - protein, 2551 aa, Homo sapiens
  PEPTIDE (1)..(2251): human FEX-2
MMLQHLVIFCLGLVVQNFCSPAETTGQARRCDRKSLLTIRTECRSCALNLGVKCPDGYTMITSGSVGVRDCRYTFEVRTYSLSLPGCRHICRKDYLQPRCCPGRWGPDCIECPGGAGSPCNGRGSCAEGMEGNGTCSCQEGFGGTACETCADDNLFGPSCSSVCNCVHGVCNSGLDGDGTCECYSAYTGPKCDKPIPECAALLCPENSRCSPSTEDENKLECKCLPNYRGDGKYCDPINPCLRKICHPHAHCTYLGPNRHSCTCQEGYRGDGQVCLPVDPCQINFGNCPTKSTVCKYDGPGQSHCECKEHYQNFVPGVGCSMTDICKSDNPCHRNANCTTVAPGRTECICQKGYVGDGLTCYGNIMERLRELNTEPRGKWQGRLTSFISLLDKAYAWPLSKLGPFTVLLPTDKGLKGFNVNELLVDNKAAQYFVKLHIIAGQMNIEYMNNTDMFYTLTGKSGEIFNSDKDNQIKLKLHGGKKKVKIIQGDIIASNGLLHILDRAMDKLEPTFESNNEQTIMTMLQPRYSKFRSLLEETNLGHALDEDGVGGPYTIFVPNNEALNNMKDGTLDYLLSPEGSRKLLELVRYHIVPFTQLEVATLISTPHIRSMANQLIQFNTTDNGQILANDVAMEEIEITAKNGRIYTLTGVLIPPSIVPILPHRCDETKREMKLGTCVSCSLVYWSRCPANSEPTALFTHRCVYSGRFGSLKSGCARYCNATVKIPKCCKGFYGPDCNQCPGGFSNPCSGNGQCADSLGGNGTCICEEGFQGSQCQFCSDPNKYGPRCNKKCLCVHGTCNNRIDSDGACLTGTCRDGSAGRLCDKQTSACGPYVQFCHIHATCEYSNGTASCICKAGYEGDGTLCSEMDPCTGLTPGGCSRNAECIKTGTGTHTCVCQQGWTGNGRDCSEINNCLLPSAGGCHDNASCLYVGPGQNECECKKGFRGNGIDCEPITSCLEQTGKCHPLASCQSTSSGVWSCVCQEGYEGDGFLCYGNAAVELSFLSEAAIFNRWINNASLQPTLSATSNLTVLVPSQQATEDMDQDEKSFWLSQSNIPALIKYHMLLGTYRVADLQTLSSSDMLATSLQGNFLHLAKVDGNITIEGASIVDGDNAATNGVIHIINKVLVPQRRLTGSLPNLLMRLEQMPDYSIFRGYIIQYNLANAIEAADAYTVFAPNNNAIENYIREKKVLSLEEDVLRYHVVLEEKLLKNDLHNGMHRETMLGFSYFLSFFLHNDQLYVNEAPINYTNVATDKGVIHGLGKVLEIQKNRCDNNDTTIIRGRCRTCSSELTCPFGTKSLGNEKRRCIYTSYFMGRRTLFIGCQPKCVRTVITRECCAGFFGPQCQPCPGNAQNVCFGNGICLDGVNGTGVCECGEGFSGTACETCTEGKYGIHCDQACSCVHGRCNQGPLGDGSCDCDVGWRGVHCDNATTEDNCNGTCHTSANCLTNSDGTASCKCAAGFQGNGTICTAINACEISNGGCSAKADCKRTTPGRRVCTCKAGYTGDGIVCLEINPCLENHGGCDKNAECTQTGPNQAACNCLPAYTGDGKVCTLINVCLTKNGGCSEFAICNHTGQVERTCTCKPNYIGDGFTCRGSIYQELPKNPKTSQYFFQLQEHFVKDLVGPGPFTVFAPLSAAFDEEARVKDWDKYGLMPQVLRYHVVACHQLLLENLKLISNATSLQGEPIVISVSQSTVYINNKAKIISSDIISTNGIVHIIDKLLSPKNLLITPKDNSGRILQNLTTLATNNGYIKFSNLIQDSGLLSVITDPIHTPVTLFWPTDQALHALPAEQQDFLFNQDNKDKLKEYLKFHVIRDAKVLAVDLPTSTAWKTLQGSELSVKCGAGRDIGDLFLNGQTCRIVQRELLFDLGVAYGIDCLLIDPTLGGRCDTFTTFDASGECGSCVNTPSCPRWSKPKGVKQKCLYNLPFKRNLEGCRERCSLVIQIPRCCKGYFGRDCQACPGGPDAPCNNRGVCLDQYSATGECKCNTGFNGTACEMCWPGRFGPDCLPCGCSDHGQCDDGITGSGQCLCETGWTGPSCDTQAVLPAVCTPPCSAHATCKENNTCECNLDYEGDGITCTVVDFCKQDNGGCAKVARCSQKGTKVSCSCQKGYKGDGHSCTEIDPCADGLNGGCHEHATCKMTGPGKHKCECKSHYVGDGLNCEPEQLPIDRCLQDNGQCHADAKCVDLHFQDTTVGVFHLRSPLGQYKLTFDKAREACANEAATMATYNQLSYAQKAKYHLCSAGWLETGRVAYPTAFASQNCGSGVVGIVDYGPRPNKSEMWDVFCYRMKDVNCTCKVGYVGDGFSCSGNLLQVLMSFPSLTNFLTEVLAYSNSSARGRAFLEHLTDLSIRGTLFVPQNSGLGENETLSGRDIEHHLANVSMFFYNDLVNGTTLQTRLGSKLLITASQDPLQPTETRFVDGRAILQWDIFASNGIIHVISRPLKAPPAPVTLTHTGLGAGIFFAIILVTGAVALAAYSYFRINRRTIGFQHFESEEDINVAALGKQQPENISNPLYESTTSAPPEPSYDPFTDSEERQLEGNDPLRTL

SEQ ID NO: 2 - DNA, 22 bp, Artificial Sequence
  Fex23 sense primer
tgccccatcgatgtgatgaaac

SEQ ID NO: 3 - DNA, 25 bp, Artificial Sequence
  Fex23 antisense primer
caaacaagagctcccgctgcacaat

SEQ ID NO: 4 - DNA, 25 bp, Artificial Sequence
  Fex45 sense primer
gcagcgggagctcttgtttgacctg

SEQ ID NO: 5 - DNA, 25 bp, Artificial Sequence
  Fex45 antisense primer
cccgtccaagcttgcacagtgtcct

SEQ ID NO: 6 - DNA, 24 bp, Artificial Sequence
  RACE-GSP-1 sense primer
tcccagcttactcagtggccaggc

SEQ ID NO: 7 - DNA, 29 bp, Artificial Sequence
  RACE-GSP-2 antisense primer
caggcccatcatatttgcacactgtagac

SEQ ID NO: 8 - DNA, 26 bp, Artificial Sequence
  RACE-GSP-3 antisense primer
agttaatttggcaggggtccacaggc

SEQ ID NO: 9 - DNA, 25 bp, Artificial Sequence
  FEX-1 sense primer
aaggcaggtctcacctatctcctgg

SEQ ID NO: 10 - DNA, 22 bp, Artificial Sequence
  FEX-1 antisense primer
tcacaaatgcatgtcccgttgc

SEQ ID NO: 11 - DNA, 25 bp, Artificial Sequence
  hFex2-4E5 sense primer
aaaaaggatccacagtgtttgctcc

SEQ ID NO: 12 - DNA, 27 bp, Artificial Sequence
  hFex2-4E5 antisene primer
ttttactcgagacttttgggagatagc

(19) United States
(12) Patent Application Publication
(10) Pub. No.: US 2009/0028871 A1
(43) Pub. Date: Jan. 29, 2009

(54) NOVEL USE OF LIGANDS SPECIFIC TO FEX-2 POLYPEPTIDE

(75) Inventors: In-San Kim, Daegu (KR); Seung-Yeon Park, Gyeongbuk (KR); Mi-Yeon Jung, Daegu (KR); Ha-Jeong Kim, Daegu (KR)

Correspondence Address: BUCHANAN, INGERSOLL & ROONEY PC, POST OFFICE BOX 1404, ALEXANDRIA, VA, 22313-1404, US

(73) Assignee: KYUNGPOOK NATIONAL UNIVERSITY INDUSTRY-ACADEMIC COOPERATION FOUNDATION, Daegu (KR)

(21) Appl. No.: 11/665,190

(22) PCT Filed: Oct. 12, 2005
(86) PCT No.: PCT/KR05/03404

§ 371 Date: Apr. 12, 2007

(30) Foreign Application Priority Data
Oct. 12, 2004 (KR) 10-2004-0081499

(51) Int. Cl. A61K 39/395; A61K 31/661; G01N 33/53; A61P 37/06

(52) U.S. Cl. 424/145.1; 514/121; 424/158.1; 435/7.1

(57) ABSTRACT

The present invention relates to novel use of a ligand specific to a FEX-2 polypeptide. More specifically, the present invention relates to methods for modulating the secretion of an inflammation-associated cytokine and for treating or preventing an inflammatory disease using the FEX-2-specific ligand. The ligand specific to the FEX-2 polypeptide can bind to FEX-2 expressed on the surface of phagocytes so that it can stimulate the secretion of anti-inflammatory cytokine and inhibit the secretion of inflammatory cytokine, so as to treat or prevent inflammatory diseases.

This application claims priority to Korean Patent Application No. 10-2004-0081499, filed on Oct. 12, 2004, the contents of which are hereby incorporated by reference.

### FIELD OF THE INVENTION

The present invention relates to novel use of a ligand specific to a FEX-2 polypeptide.

### BACKGROUND OF THE INVENTION

Inflammation is a local or systemic protective response to the injury or infection of cells or tissues. Inflammation is typically caused by a series of biological reactions occurring due to the direct reaction of a large number of humoral immune mediators that consist of immune system or the stimulation of the local or systemic effector system by the mediators. The mediators involved in such inflammatory reactions include immune cells, such as polymorphonuclear leukocytes (PMN), cytotoxic T lymphocyte (CTL), natural killer (NK) cells and macrophages etc, and cytokines. Inflammatory diseases are caused particularly by an imbalance of inflammatory cytokines and by the interaction between effector cells. Main inflammatory diseases include: rhinitis and sinusitis such as infectious rhinitis, allergic rhinitis, chronic rhinitis, acute sinusitis and chronic sinusitis; otitis media such as acute purulent otitis media and chronic purulent otitis media; pneumonia such as bacterial pneumonia, bronchial pneumonia, lobar pneumonia, Legionella pneumonia and viral pneumonia; acute or chronic gastritis; enteritis such as infectious enterocolitis, Crohn's disease, idiopathic ulcerative colitis and pseudomembranous colitis; arthritis such as septic arthritis, tuberculous arthritis, degenerative arthritis and rheumatoid arthritis; and diabetic ocular disease.

Main inflammatory cytokines known up to now include tumor necrosis factor-α (TNF-α), IL-1, IL-6 and IL-18, which are produced by monocytes and macrophages. Among them, TNF-α performs suitable homeostatic and protective actions at low concentrations, but aggravates inflammatory reactions at high concentrations by interaction with other kinds of inflammatory cytokines, such as IL-1, in systemic tissue or a specific tissue.

Meanwhile, TGF-β is a cytokine having the activity of stimulating or inhibiting a certain cell, and has activities, such as an antagonistic effect on lymphocyte response, and the inhibition of macrophage activation. For this reason, TGF-β is known as a typical anti-inflammatory cytokine.

To treat inflammatory diseases caused by an imbalance of inflammatory cytokines, various studies to inhibit the inflammatory cytokines and stimulate the production of anti-inflammatory cytokines have been conducted.

Methods of inhibiting proinflammatory cytokine include a production inhibitory method for inhibiting the secretion of cytokines from being secreted by external stimulation, and a functional inhibitory method for modulating the stimulation of target cells caused by cytokines. Those known as inhibitors of cytokine production include SK&F 86002, which is inhibitors of IL-1 and TNF-α production, tetradrine and WIN 67694 and those developed as inhibitors of cytokine function include IL-1 receptor antagonist IL-1Ra, soluble TNF-α receptors and cytokine antibodies.

### DETAILED DESCRIPTION OF THE INVENTION

### Technical Problem

Therefore, it is an object of the present invention to provide novel use of a ligand specific to FEX-2.

### Technical Solution

Accordingly, the present inventors have conducted many studies to develop a novel therapeutic agent for inflammatory diseases and as a result, found that a ligand specific to FEX-2 can bind to FEX-2 expressed on the surface of phagocytes to stimulate the secretion of anti-inflammatory cytokine and to inhibit the secretion of inflammatory cytokine, thereby treating or preventing inflammatory diseases.

To achieve the above object, in one aspect, the present invention provides a method for modulating the secretion of an inflammation-related cytokine in a subject and a method for treating or preventing an inflammatory disease, the methods comprising administering to FEX-2 the subject in need thereof an effective amount of a ligand specific to FEX-2.

In another aspect, the present invention provides a method for screening an agent for modulating an inflammation-associated cytokine, the method comprising the steps of: (a) culturing a test agent with phagocyte expressing FEX-2; and (b), measuring change in the level of cytokine secreted from the phagocyte cultured with the test agent relative to the level of cytokine secreted from the phagocyte cultured without the test agent.

In still another aspect, the present invention provides a method for identifying an agent for treating or preventing of an inflammatory disease, the method further comprising administering the test agent selected in step (b), which modulates the level of cytokine secreted from phagocyte, to an animal having inflammatory diseases, and determining whether the test agent shows a therapeutic effect in the animal, in addition to the steps of the screening method.

In yet another aspect, the present invention provides the uses of a ligand specific to a FEX-2 polypeptide, for preparing a pharmaceutical composition for modulating the secretion of an inflammation-associated cytokine and a pharmaceutical composition for the treatment or prevention of an inflammatory disease.

In still yet another aspect, the present invention provides a pharmaceutical composition for modulating an inflammation-associated cytokine and a pharmaceutical composition for the treatment or prevention of an inflammatory disease comprising, as an active ingredient, a ligand specific to a FEX-2 polypeptide.

### DEFINITIONS

Unless defined otherwise, all technical and scientific terms used herein have the same meanings as commonly understood by those ordinary skill in the art to the present invention pertains. The following references provide one of skill with a general definition of many of the terms used in the present invention: Singleton et al., DICTIONARY OF MICROBIOLOGY AND MOLECULAR BIOLOGY (2^{nd} ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY. In addition, the following definitions are provided to assist the reader in the practice of the present invention.

As used herein, the term “polypeptide” is used interchangeably with the terms “polypeptides” and “protein(s)”, and refers to, a polymer of amino acid residues, e.g., as typically found in proteins in nature.

The term “FEX-2 polypeptide” may be derived from mammals, and may preferably be derived from any one selected from the group consisting of human beings, rats and mice. More preferably, it may be human FEX-2 comprising the amino acid sequence shown in SEQ ID NO: 1. Most preferably, the FEX-2 may be one expressed in mammalian phagocytes.

The term “ligand specific to FEX-2” is meant to include all kinds of peptides, polypeptides, peptide imitations, compounds and biological preparations, which bind to FEX-2. Preferably, the FEX-2-specific ligand refers to one having an activity capable of binding to FEX-2 expressed on the surface of phagocytes so as to modulate the secretion of cytokine from the activated phagocytes. More preferably, the FEX-2-specific ligand may be one having activities capable of stimulating the secretion of anti-inflammatory cytokines from phagocytes and inhibiting the secretion of inflammatory cytokine from phagocytes. Examples of the phagocytes include not only macrophages which are professional phagocytes, but also non-professional phagocytes, such as epithelial cells and fibroblasts. Most preferably, the FEX-2-specific ligand may be phosphatidylserine, an anti-FEX-2 antibody, or derivatives thereof. As used herein, the term “derivatives” refers to those obtained by modifying a part of the chemical structure of the phosphatidylserine or anti-FEX-2 antibody while maintaining the fundamental framework and physiological activity thereof.

The phosphatidylserine is a kind of naturally occurring lipid and consists of a serine, phosphate, glycerol and two fatty acids. After the phosphatidylserine was first isolated by Folch in 1948, it has been reported to have various effects, such as the treatment of dementia, the improvement of brain function, the treatment of epilepsy, and stress resistance by a great many research studies. The phosphatidylserine used in the present invention is not specifically limited and may be derived from nature or be synthesized or be commercially available. Also, the phosphatidylserine derivative may be preferably phospho-L-serine. The phosphatidylserine or derivatives thereof may be in the form of powder, granule, paste, liquid or salt. As the salt, any salt may be used if it is pharmaceutically-acceptable. Examples of the pharmaceutically acceptable salt may include sodium salt, potassium salt, magnesium salt, ammonium salt, phosphate salt, chloride salt and sulfate salt.

Also, the anti-FEX-2 antibody, as used herein, may be a polyclonal or monoclonal antibody. The antibody used in the present invention may be prepared using a FEX-2 protein as an antigen by any conventional method widely known in the immunology field.

The polyclonal antibody may be prepared from warm-blooded animals, such as horses, cattle, goats, sheep, dogs, fowls, turkeys, rabbits, mice or rats, according to any conventional technology known in the art. Namely, animals are immunized by the intraperitoneal, intramuscular, intraocular or subcutaneous injection of the antigen. Immunity to the antigen can be increased by the use of an adjuvant, such as Freund's complete or incomplete adjuvant. After booster immunization, a small sample of serum is obtained and tested for reactivity to the desired antigen. Once the titer of the animal has reached a plateau in views of its reactivity to the antigen, large amounts of polyclonal immune serum may be readily obtained either by weekly bleeding or by exsanguinating the animal.

The monoclonal antibody may also be readily generated using well-known techniques (see Kennet™, McKearn, and Bechtol (eds.), Monoclonal Antibodies, Hybridomas; A New Dimension in Biological Analyses, Plenum Press, 1980). Specifically, the monoclonal antibody may be prepared by immunizing an animal using a protein as an immunogen; fusing the spleen cells of the immunized animal with myeloma cells to produce hybridomas; selecting from the produced hybridomas a hybridoma selectively recognizing the FEX-2 protein; culturing the selected hybridoma; and isolating the desired antibody from the hybridoma culture medium. Alternatively, the monoclonal antibody used in the present invention may also be prepared by injecting into an animal the hybridoma producing the anti-FEX2 antibody, which selectively recognizes the FEX-2 protein; and isolating the antibody from the collected abdominal dropsy of the animal at a given period after the injection. The hybridoma 5G3 producing the human FEX-2 monoclonal antibody, prepared in one example of the present invention, was deposited under accession No. KCTC-10639BP on May 21, 2004 with the Korean Collection for Type Cultures (KCTC), Korean Research Institute of Bioscience and Biotechnology, (52, Oun-dong, Yusong-ku, Taejon, Korea), which is an International Depository Authority under the Budapest Treaty. The deposit shall be maintained in viable condition at the KCTC during the entire term of the issued patent and shall be made available to any person or entity for non-commercial use without restriction, but in accordance with the provisions of the law governing the deposit.

Derivatives of the anti-FEX-2-antibody according to the present invention may include those obtained by modifying the structure of the antibody to alter the stability, storage stability, volatility or solubility of the antibody. In some cases; they may be those obtained by modifying the antibody by phosphorylation, sulfation, acrylation, glycosylation, methylation, farnesylation, etc.

As used herein, the term “subject” may be an animal, and is preferably a mammal. It may also be cells, tissues, organs, etc., derived from the animals.

As used herein, the term “effective amount” refers to an amount that exhibits the effect of modulating the secretion of an inflammation-associated cytokine in a subject, and preferably, the effect of modulating the secretion of cytokine from phagocytes, and most preferably, the effect of stimulating the secretion of anti-inflammatory cytokine and/or inhibiting the secretion of inflammatory cytokine, or thus, the effect of treating or preventing an inflammatory disease.

As used herein, the term “agent” or “test agent” includes any substance, molecule, element, compound, entity, or a combination thereof. It includes, but is not limited to e.g., protein, polypeptide, small organic molecule, polysaccharide and polynucleotide. Moreover, it may also be a natural product, a synthetic compound, a chemical compound, or a combination of two or more substances. Unless otherwise specified, the terms “agent”, “substance” and “compound” can be used interchangeably.

Hereinafter, the present invention will be described in detail.

The present inventors searched for partial human cDNA sequences comprising fas-1 domain in the known nucleotide database. Among the searched sequences, cDNA sequences whose characteristics are not yet identified were selected. On the basis of the cDNA sequences, primers were designed, and then, RT-PCR and 5′ RACE PCR were performed using total RNA extracted from the human spleen as a template, thus cloning a new human gene comprising the fas-1 domain (see Example 1).

The gene synthesized by the present inventors had seven fas-1 domains, twenty-three EGF-like domains, one X-link domain and one transmembrane domain (see FIG. 1). On the basis of the domain structure as described above, the gene was named “FEX-2” and its gene sequence was registered in GeneBank under accession number AY311388. The full amino acid sequence of the human FEX-2 gene is set forth in SEQ ID NO: 1.

The present inventors conducted various tests using fibroblasts transformed with the FEX-2 gene to examine the function of the FEX-2 gene. As a result, we have found that the FEX-2 specifically recognizes phosphatidylserine expressed on the surface of aged cells and apoptotic cells, thereby mediating the binding and phagocytosis of the cells. Also, the present inventors have found that the FEX-2 gene is expressed on the surface of macrophages involved in the phagocytosis of aged cells and apoptotic cells, and the phagocytosis by macrophages is mediated by the FEX-2 gene. From this fact, the present inventors confirmed that the FEX-2 gene is a novel receptor for phosphatidylserine.

Furthermore, the present inventors have found that, when macrophages (activated phagocytes) or fibroblasts transformed with FEX-2 was treated with a ligand specific to FEX-2, the secretion of anti-inflammatory cytokines would be stimulated and the secretion of inflammatory cytokines would be inhibited. This suggests that the FEX-2-specific ligand can be used as an agent for modulating the secretion of an inflammation-associated cytokines in a subject and an agent for the prevention or treatment of an inflammatory disease.

More specifically, in one embodiment of the present invention, a recombinant vector comprising the FEX-2 gene was prepared and transfected into L cells (mouse fibroblasts), thus preparing L/FEX-2 cells (see Example 1). Then, an examination was made to check whether or not the prepared cells are involved in the binding and phagocytosis of aged cells and apoptotic cells (see Example 4). As a result, it could be seen that the L/FEX-2 cells selectively recognized, and bind to the aged red blood cells, thereby phagocytising them (see FIGS. 3 and 4). This suggests that the binding and phagocytosis of the aged red blood cells by L/FEX-2 cells is mediated by FEX-2.

Furthermore, in another embodiment of the present invention, the recognition sites of FEX-2 in the binding of aged cells to FEX-2 were identified (see Example 5).

As a result, it could be found that, as aging progressed, the expression of phosphatidylserine on the surface of red blood cells increased (see FIG. 5), and the binding of red blood cells to L/FEX-2 cells also increased (see FIG. 6). In addition, it could be found that the FEX-2 gene selectively recognized only the phosphatidylserine on the surface of red blood cells (see FIGS. 7 to 9), and the FEX-2 gene did not recognize phosphatidylserine through negative charge, but rather specifically recognized the structure of phosphatidylserine (see FIG. 10).

Also, in view of the fact that phosphatidylserine is expressed on the surface of not only aged cells but also apoptotic cells, the present inventors examined whether apoptotic cells are phagocytised by the L/FEX-2 cells (see Example 6). As a result, it could be found that L/FEX-2 cells recognized phosphatidylserine expressed on the surface of not only aged cells but also apoptotic cells thereby mediating the binding and phagocytosis of these cells (see FIGS. 12 and 13).

Meanwhile, the present inventors treated the L/FEX-2 cells with the FEX-2 monoclonal antibody and measured the secretion of anti-inflammatory cytokine from the cells (see Example 6). As a result, it could be found that the treatment of the L/FEX-2 cells with the FEX-2 monoclonal antibody resulted in an increase in the secretion of the anti-inflammatory cytokine (see FIG. 14).

Also, since the phagocytosis of aged cells and apoptotic cells is mainly undertaken by macrophages, the present inventors examined whether the FEX-2 is expressed on the surface of macrophages and whether FEX-2 is involved in the phagocytosis by macrophages (see Example 7). As a result, it could be found that the FEX-2 was expressed on macrophages (see FIG. 15), and the phagocytosis by macrophages was made as a result of a process where the FEX-2 existing on the surface of macrophages specifically recognized phosphatidylserine expressed on apoptotic cells (see FIG. 16). This indicates that the FEX-2 is a novel receptor for phosphatidylserine.

It is known that the phosphatidylserine-dependent phagocytosis of apoptotic cells stimulates the secretion of anti-inflammatory cytokine TGF-β and inhibits the secretion of inflammatory cytokine TNF-α. In view of this fact, the present inventors examined whether a ligand specific to the FEX-2 existing on the surface of macrophages can transfer to activated macrophages the same signal as a signal generated during the phagocytosis of macrophages thereby stimulate the secretion of anti-inflammatory cytokine (see Example <7-3>). As a result, it could be found that macrophages activated by treatment with the phosphatidylserine or the monoclonal antibody to FEX-2 stimulated the secretion of anti-inflammatory cytokine and inhibited the secretion of inflammatory cytokine, similarly to the case where macrophages phagocytise the aged cells (see FIG. 17).

Therefore, the present invention provides a method for modulating the secretion of an inflammation-associated cytokine in a subject and a method for treating or preventing an inflammatory disease, the methods comprising administering to a subject in need thereof an effective amount of a ligand specific to the FEX-2 polypeptide. In the method for modulating the secretion of an inflammation-associated cytokine, the cytokine may be secreted from phagocytes. The cytokine includes all anti-inflammatory cytokines and inflammatory cytokines. For example, the anti-inflammatory cytokine may be selected from the group consisting of TGF-β, IL-10, IL-4 and IL-13, but not limited thereto (Watanabe, M. et al., Adeno-associated virus-mediated human IL-10 gene transfer suppresses the development of experimental autoimmune orchitis. Gene Therapy 2005, 12:1126-1132; Ghoreschi; K. et al., Interleukin-4 therapy of psoriasis induces Th2 responses and improves human autoimmune disease. Nature Medicine 2003, 9:40-46; Kluth, D. C. et al., New approaches to modify glomerular inflammation. J. Nephrol. 1999, 12:66-75), and may preferably be TGF-β. Also, the inflammatory cytokine may be selected from the group consisting of TNF-α, IL-1, IL-6, IL-8, IL-18 and MIP₂, but not limited to (see McDonald, P. P. et al., Transcriptional and translational regulation of inflammatory mediator production by endogenous TGF-13 in macrophages that have ingested apoptotic cells. J. immunol. 1999, 1, 63:6164-6172; Dinarello, C. A. et al., Blocking IL-1 in systemic inflammation. J Exp Med. 2005, 201:1355-9; de Boer, W. I. et al., Perspectives for cytokine antagonist therapy in COPD. Drug Discov Today. 2005, 10:93-106), and may preferably be TNF-α.

In the above method, modulating the secretion of cytokines may preferably be stimulating the secretion of anti-inflammatory cytokine and/or inhibiting the secretion of inflammatory cytokine. For example, stimulating the secretion of TGF-β and inhibiting the secretion of TNF-α may be employed.

The ligand specific to the FEX-2 polypeptide, which is used in the inventive method, can be provided in the form of a pure substance or a suitable pharmaceutical composition. The pharmaceutical composition is characterized by comprising the ligand specific to FEX-2, as an active ingredient. The pharmaceutical composition may be prepared by formulating the inventive FEX-2-specific ligand into a suitable form together with a pharmaceutically acceptable carrier. As used herein, the term “pharmaceutically acceptable carrier” refers to a composition which is physiologically acceptable and, when administered to human beings, generally does not cause allergic reactions such as gastrointestinal disorder and dizziness, or similar reactions thereto. Example of the pharmaceutically acceptable carrier includes oral carrier such as lactose, starch, cellulose derivative, magnesium stearate, stearic acid, etc; and parenteral carrier such as water, suitable oil, saline solution, aqueous glucose, glycol etc. The composition may further comprise a stabilizer and a preservative. Suitable stabilizer includes an antioxidant, such as sodium bisulphite, sodium sulphite or ascorbic acid. Suitable preservative includes benzalkonium chloride, methylparaben, propylparaben and chlorobutanol. Other pharmaceutically acceptable carriers can be found in the following literature: Remington's Pharmaceutical Sciences, 19th ed., Mack Publishing Company, Easton, Pa., 1995.

The inventive pharmaceutical composition can be prepared in the form of various parenteral or oral forms according to a conventional method. The parenteral formulation typically includes an injection formulation, and preferably an isotonic solution or a suspension. The injection formulation may be prepared using a suitable dispersing agent, wetting agent or suspending agent according to any method known in the art. For example, the injection formulation can be prepared by dissolving necessary components in a saline or buffer solution. Also, the oral formulation includes, but are not limited to, powder, granule, tablet, pill and capsule.

The pharmaceutical composition formulated as described above may be administered in an effective amount by various routes, including oral, transdermal, subcutaneous, intravenous and intramuscular routes.

The dose of either the inventive ligand specific to the FEX-2 polypeptide or the pharmaceutical composition comprising the ligand as an active ingredient may be suitably selected depending on an administration route, subject and its age, body weight, sex, disease severity. Although the dose of the active ingredient may vary depending on disease severity, the active ingredient may preferably be generally administered several times a day at a one time effective dose of 10 μg-10 mg for adult.

The inventive pharmaceutical composition prepared as described above can be effectively used for modulating the secretion of an inflammation-associated cytokine in a subject and for preventing and treating an inflammatory disease. As used herein, the term “inflammatory disease” is meant to include simple inflammation itself and all diseases caused by the inflammatory reaction. Examples of the inflammatory diseases include, but are not limited to, inflammation, inflammatory bowl disease, diabetic ocular-disease, peritonitis, osteomyelitis, cellulitis, meningitis, encephalitis, pancreatitis, trauma causing shock, bronchial asthma, rhinitis, sinusitis, otitis media, pneumonia, gastritis, enteritis, cystic fibrosis, apoplexy, bronchitis, bronchiolitis, hepatitis, nephritis, arthritis, gout, spondylitis, Reiter's syndrome, polyarteritis nodosa, hypersensitivity vasculitis, Wegener's granulomatosis, polymyalgia rheumatica, giant cell arteritis, calcium crystal deposition arthropathy, pseudogout, nonarticular rheumatism, bursitis, tenosynovitis, epicondylitis (Tennis elbow), neuropathic joint disease (Charcot's joint), hemarthrosis, Henoch-Schonlein Purpura, hypertrophic osteoarthropathy, multicentric reticulohistiocytoma, scoliosis, hemochromoatosis, sickle cell disease and other hemoglobinopathies, hyperlipoproteinemia, hypogammaglobulinemia, hyperparathyroidism, acromegaly, familial mediterranean fever, Behcet's disease, systemic lupus erythematosus, relapsing fever, psoriasis, multiple sclerosis, septicemia, septic shock, acute respiratory distress syndrome, multiple organ failure, chronic obstructive pulmonary disease, acute lung injury and broncho-pulmonary dysplasia.

Also, the present invention provides methods for screening an agent for modulating the secretion of an inflammation-associated cytokine in phagocyte and an agent for treating and preventing an inflammatory disease.

Specifically, the screening methods comprise:

(a) culturing a phagocyte expressing FEX-2 with a test agent; and

(b) measuring change in the level of cytokine secreted from the phagocyte cultured with the test agent relative to the level of cytokine secreted from phagocytes cultured without the test agent.

In the screening method, the phagocyte expressing FEX-2 in the step (a) is preferably macrophage or mouse fibroblast L cell transformed with FEX-2. Also, the step (b) can be performed by measuring the production of the anti-inflammatory cytokine (e.g., TGF-β) and/or inflammatory cytokine (e.g., TNF-α) using an ELISA method known in the art.

Furthermore, the method for screening the agent for treating or preventing an inflammatory disease may comprises, in addition to the steps (a) and (b), the step of: (c) administering the test agent selected in step (b), which modulates the level of cytokine secreted from phagocyte, to an animal suffering from inflammatory disease, and determining whether the test agent shows a therapeutic effect in the animal.

The term “animal” used in the step (c) is preferably a non-human animal. Also, the term “therapeutic effect” used in the step (c) means the effect of alleviating or improving an inflammatory disease and its symptoms and the effect of inhibiting the progression of the inflammatory disease.

Agents which can be screened according to the present invention may preferably be either agents for stimulating the secretion of anti-inflammatory cytokine and/or inhibiting the secretion of inflammatory cytokine, or agents showing the effect of treating or treating the inflammatory disease through these actions.

Test agents which can be screened according to the inventive methods include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, saccharides, fatty acids, purins, pyrimidines, derivatives, structural analogs or combinations thereof. Some test agents may be synthetic molecules, and others natural molecules. The test agents can be obtained from a wide variety of source, including libraries of synthetic or natural compounds. Combinatorial libraries can be produced for many types of compounds that can be synthesized in a step-by-step fashion. Large combinatorial libraries of compounds can be constructed by the encoded synthetic libraries (ESL) method described in WO 95/12608, WO 93/06121, WO 94/08051, WO 95/395503 and WO 95/30642. Libraries of natural compounds in the form of bacterial, fungal, plant and animal extracts can be obtained from commercial sources or collected in the field. Additionally, known pharmacological agents can be subject to directed or random chemical modifications, such as acylation, alkylation, esterification, amidification, etc. to produce structural analogs. The test agents may be naturally occurring proteins or their fragments. Such test agents can be obtained from natural sources, for example, a cells or tissue lysate. Libraries of polypeptides agents can also be prepared, e.g., from a cDNA library commercially available or generated with routine methods. The test agents can also be peptides, e.g., peptides having about 5-30 amino acids, preferably about 5-20 amino acids, and more preferably about 7-15 amino acids. The peptides can be digests of naturally occurring proteins, random peptides, or “biased” random peptides.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

The test agents may be small molecules (e.g., molecules with a molecular weight of not more than about 1,000). Preferably, high throughput assays are adapted and used to screen for such small molecules. In some cases, combinatorial libraries of small molecule test agents as described above can be readily employed in the inventive screening method (Shultz, Bioorg. Med. Chem. Lett., 8:2409-2414, 1998; Weller, Mol. Drivers., 3:61-70, 1997; Fernandes, Curr. Opin. Chem. Biol., 2:597-603, 1998; and Sittampalam, Curr. Opin. Chem. Biol., 1:384-91, 1997).

Libraries of test agents, to be screened with the inventive methods, can also be generated based on structural studies of the FEX-2 polypeptide or a ligand specific thereto, or fragments or analogues thereof. The term “analogue” as used herein refers to a molecule that structurally resembles a reference molecule but which has been modified in a targeted and controlled manner, by replacing a specific substituent of the reference molecule with an alternate substituent. Compared to the reference molecule, an analogue would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

Such structural studies allow the identification of test agents that are likely to bind to the FEX-2 polypeptide. The three-dimensional structure of the FEX-2 polypeptide can be studied in a number of ways, e.g., crystal structure and molecular modeling. Methods of studying protein structures using X-ray crystallography are well known in the following literatures: Physical Bio-chemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of the structure of FEX-2 provides another means for designing test agents for screening an agent for modulating the secretion of an inflammation-associated cytokine. Methods of molecular modeling are described in the following literature: U.S. Pat. No. 5,612,894 and U.S. Pat. No. 5,583,973. In addition, protein structures can also be determined by neutron diffraction and nuclear magnetic resonance (NMR). See Physical Chemistry, 4^{th} Ed. Moore, W. J. (Prentice-Hall, New Jersey 1972) and NMR of Proteins and Nucleic Acids, K. Wuthrich (Wiley-Interscience, New York 1986).

### BRIEF DESCRIPTION OF THE DRAWINGS

FIG. 1 is a schematic diagram showing each domain of a human FEX-2 protein.

FIG. 2 shows the results of flow cytometry analysis using a FEX-2 monoclonal antibody, conducted in order to examine whether FEX-2 is expressed on the surface of L/FEX-2 cells. Control group: mouse immunoglobulin.

FIG. 3 shows the binding and phagocytosis of aged cells and normal red blood cells by L/FEX-2 cells, compared to those for normal red blood cells and L/Mock cells.

FIG. 4 shows the inhibitory effect of the FEX-2 monoclonal antibody on the binding and phagocytosis of aged red blood cells,

None: untreated L/FEX-2 cells

5G3: L/FEX-2 cells pre-cultured with the FEX-2 monoclonal antibody

IgG1: L/FEX-2 cells pre-cultured with mouse immunoglobulin

FIG. 5 shows the results of flow cytometric measurement using an annexin V antibody, conducted to measure the expression of phosphatidylserine according to the aging of red blood cells.

FIG. 6 shows measurement results for the binding of red blood cells to L/FEX-2 cells according to the aging of red blood cells.

FIG. 7 is a graphic diagram showing the binding of various phospholipids to L/FEX-2 cells.

PI: phosphatidylionsitol;

PE: phosphatidylethanolamine;

PS: phosphatidylserine; PC: phosphatidylcholine;

PA: phosphatidic acid; and

PG: phosphatidylglycerol.

FIG. 8 is a graphic diagram showing the inhibition of the binding and phagocytosis of aged red bloods by L/FEX-2 cells according to the treatment of various phospholipids.

None: no addition of phospholipids;

PI: phosphatidylionsitol;

PE: phosphatidylethanolamine;

PS: phosphatidylserine; PC: phosphatidylcholine;

PA: phosphatidic acid; and

PG: phosphatidylglycerol.

FIG. 9 is a graphic diagram showing the binding of aged red blood cells to L/FEX-2 cells according to the concentration of phosphatidylserine or phosphatidylcholine.

FIG. 10 shows the inhibitory effect of structural analogues of phosphatidylserin on the binding and phagocytosis of aged red cells by L/FEX-2 cells.

None: no addition of phospholipids;

PS: phosphatidylserine;

PDS: phospho-D-serine; and

PLS: phospho-L-serine.

FIG. 11 shows the results of flow cytometry analysis for apoptosis in Jurkat T cells treated with an anti-Fas-antibody and U937 cells treated with ectoposide.

FIG. 12 illustrates photographs showing the phagocytosis of apoptotic cells by L/FEX-2 cells and L/Mock cells.

a: L/Mock cells; and

b: L/FEX-2 cells.

FIG. 13 is a graphic diagram showing the inhibitory effect of phosphatidylserine or a FEX-2 monoclonal antibody on the phagocytosis of apoptotic cells by L/FEX-2 cells.

None: untreated;

PS: phosphatidylserine; PC: phosphatidylcholine;

5G3: FEX-2 monoclonal antibody; and

IgG1: mouse immunoglobulin.

FIG. 14 shows measurement results for the secretion of anti-inflammatory cytokine by the treatment of L/FEX-2 cells with the FEX-2 monoclonal antibody.

Con: untreated control group;

IgG: treated with mouse immunoglobulin (IgG1); and

5G3: treated with the FEX-2 monoclonal antibody.

FIG. 15 shows the results of flow cytometric analysis using the FEX-2 monoclonal antibody, conducted to examine whether FEX-2 is expressed on the surface of macrophages. Mouse immunoglobulin (IgG1) was used as a control group.

FIG. 16 is a graphic diagram showing the inhibitory effect of phosphatidylserine or FEX-2 monoclonal antibody on the apoptosis and phagocytosis by macrophages.

PS: phosphatidylserine;

IgG: mouse immunoglobulin; and

5G3: FEX-2 monoclonal antibody.

FIG. 17 shows measurement results for the secretion of anti-inflammatory cytokine and inflammatory cytokine by the treatment of an LPS-stimulated J774 macrophage cell line with various ligands.

Control group: untreated;

LPS: stimulated with LPS;

LPS+AR: stimulated with LPS and then treated with aged red blood cells;

LPS+PC: stimulated with LPS and then treated with phosphatidylcholine liposome;

LPS+PS: stimulated with LPS and then treated with phosphatidylserine liposome

LPS+IgG: stimulated with LPS and then treated with mouse immunoglobulin; and

LPS+5G3: stimulated with LPS and then treated with FEX-2 monoclonal antibody.

### BEST MODE FOR CARRYING OUT THE INVENTION

Hereinafter, the present invention will be described in detail by example. It is to be understood, however, that these examples are given for illustrative purpose only and are not construed to limit the scope of the present invention.

### Example 1

### Cloning of Human FEX-2 cDNA

<1-1> Preparation of Expression Vector

To identify a novel cell adhesion molecule comprising the fas-1 domains, sequences comprising the fas-1 domains were searched in the nucleotide database of Genbank and Celera genomics. As a result, human partial cDNA clones (FLJ00112, DZKZp434E0321, and CD44-like precursor FELL), which are not yet characterized, were selected. The full-length cDNA sequences were prepared from human spleen cells by RT-PCR and 5′ RACE PCR.

For this purpose, based on the above three kinds of searched partial cDNA clones, which comprise the fas-1 domains, two pairs of primers (SEQ ID NOS: 2-5) were first designed (see Table 1). Using 2 μg of RNA extracted from the human spleen, as a template, and each of the prior pairs of primers designed as described above, reverse transcriptase polymerase chain reaction (RT-PCR) was performed to obtain two protein partial cDNAs comprising the fas-1 domains. The PCR reaction was performed in the Expand high fidelity PCR system (Roche) under the following condition: 2 min at 95° C.; and 30 cycles of 30 sec at 94° C., 30 sec at 60° C. and 30 sec at 72° C.

As a result, 3.6 kb and 2.0 kb amplification products were obtained. Among the amplification products, the 3.6 kb cDNA was digested with restriction enzymes ClaI and SacI (TaKaRa) and then inserted into the same restriction enzyme sites of a pBluescript-KS(+) vector (Stratagene) using T4 ligase (Invitrogen), thus preparing recombinant vector “pBS-FEX23”. Then, among the amplification products, the 2.0 kb cDNA was digested with restriction enzymes SacI and HindIII (TaKaRa) and then inserted into the same restriction enzyme sites of a pET-29b vector (Novagen) using T4 ligase (Invitrogen), thus preparing recombinant vector “pET-Fex45”. Then, to combine the two cDNA sequences with each other, a fragment obtained by digesting pET-Fex45 with EcoRI was treated with a klenow enzyme to make a blunt fragment. The blunt fragment was digested with SacI and cloned into the same restriction enzyme site of the recombinant vector PET-Fex23, thus preparing recombinant vector “pET-Fex2345”.

The 5′-terminus of the cDNA clone comprising the fas-1 domain was prepared in the following manner. RNA extracted from the human spleen was amplified using the primer (SEQ ID NO: 6) shown in Table 1 below to prepare cDNA. The cDNA template was subjected to 5′ RACE PCR using the primers (SEQ ID NOS: 7 and 8) shown in Table 1 and an adaptor primer provided in the 5′ RACE system (5′ RACE system for Rapid Amplification of cDNA Ends version 2.0; Invitrogen), according to the manufacturer's instruction, thus obtaining an amplification product. Then, the amplification product was analyzed in a base sequencer (Applied Biosystems AB13700) to identify the base sequence of the 5′-terminus of FEX-2. Based on the base sequence of the 5′-terminus, primers were constructed. Using 2 μg of an RNA template extracted from the human spleen and the primer pair (SEQ ID NO: 9 and SEQ ID NO: 10) designed based on the 5′-terminus, a reverse transcriptase polymerase chain reaction (RT-PCR) was performed to obtain a 2.5 kb 5′-terminal cDNA product. The amplification product was digested with restriction enzymes EcoRI and ClaI (TaKaRa) and inserted into the same restriction enzyme sites of a pBluescript-KS(+) vector (Stratagene), thus preparing recombinant vector “pBS-Fex1”. In order to clone a complete base sequence into an expression vector, the above-prepared pET-Fex2345 was first digested with KpnI and HindIII the same restriction enzyme sites of a pcDNA3.1(−)/Myc-His vector (Invitrogen), thus preparing “pcDNA-Fex45”. Also, the pET-Fex2345 was digested with restriction enzymes BamHI and KpnI and inserted into the same restriction enzyme sites of the pcDNA-Fex45, thus preparing “pcDNA-Fex2345”. Then, the pBS-Fex1 was digested with EcoRI and ClaI and inserted into the same restriction enzyme sites of the pcDNA-Fex2345, thus cloning a complete base sequence.

The above-prepared plasmid was analyzed in a base sequencer (Applied Biosystems AB13700), and the identified base sequence was registered in Genebank™ under accession number AY311388. The above-prepared expression vector comprising the complete base sequence of FEX-2 was named “pcDNA-Fex2”.

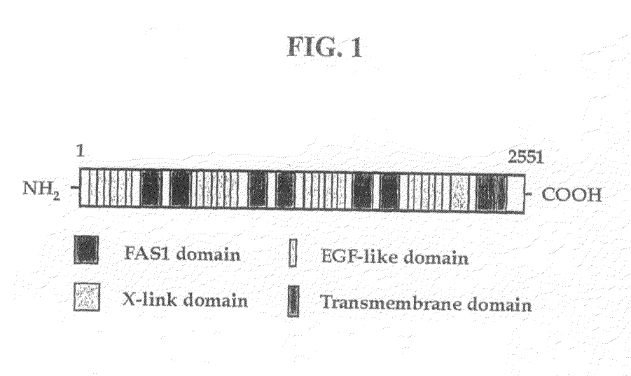

The amino acid sequence deduced from the result of the base sequence analysis was shown to be substantially identical to scavenger receptor FEEL-2 (Adachi H. et al., J. Biol. Chem. 277:34264-34270, 2002) and encocytic hyaluronan receptor stabilin-2 (Politz O. et al., Biochem J. 362:155-164, 2002) and had seven fas-1 domains, twenty-three EGF-like domains, one X-link domain and one transmembrane domain (see FIG. 1).

TABLE 1

Primers used in cloning
of Fex-2 expression vector

| | | |
|---|---|---|
| | | SEQ ID |
| Primers | Sequences | NO |

| | | |
|---|---|---|
| Fex23 | 5′-tgc ccc atc gat gtg atg aaa | 2 |
| sense | c-3′ | |
| Fex23 | 5′-caa aca aga gct ccc gct gca | 3 |
| antisense | caa t-3′ | |
| Fex45 | 5′-gca gcg gga gct ctt tga cct | 4 |
| sense | g-3′ | |
| Fex45 | 5′-ccc gtc caa gct tgc aca gtg | 5 |
| antisense | tcc t-3′ | |
| RACE-GSP-1 | 5′-tcc cag Ctt act cag tgg cca | 6 |
| antisense | ggc-3′ | |
| RACE-GSP-2 | 5′-cag gcc cat cat att tgc aca | 7 |
| antisense | ctg tag ac-3′ | |
| RACE-GSP-3 | 5′-agt taa ttt ggc agg ggt cca | 8 |
| antisense | cag gc-3′ | |
| Fex1 sense | 5′-aag gca ggt ctc acc tat ctc | 9 |
| | ctg g-3′ | |
| Fex1 | 5′-tca caa atg cat gtc ccg ttg | 10 |
| antisense | c-3′ | |

<1-2> Transformation of L Cells

Mouse fibroblast L-cells (ATCC CCL-1; provided by Dr. M. Takeichi, Department of Biophysics, Kyoto University, Japan) were transformed with the expression vector pcDNA-Fex2 prepared in Example <1-1>. The L cells were cultured in Dulbecco's modified Eagle's medium (DMEM) supplemented with 10% heat-inactivated FBS, penicillin G and streptomycin.

To transform the above-selected L cells with the recombinant vector pcDNA-Fex2, lipofectamine (Invitrogen) was used according to the manufacturer's instruction. 48 hours after the transformation, the cells were treated with G418 (400 μg/ml) and cultured for 10 to 12 days, while each colony showing resistance was isolated. The cells transformed as described above were named “L/FEX-2”. As a negative control group (L/Mock), cells transformed with pcDNA3.1 (−)/Myc-His, a vector comprising no FEX-2 gene were used.

### Example 2

### Preparation of Monoclonal Antibody to Human FEX-2

In order to prepare a monoclonal antibody to human FEX-2, cDNA comprising a base sequence corresponding to amino acid residues 1173-1727 of the entire amino acid sequence of human FEX-2 was prepared. The cDNA was obtained by performing PCR using the pcDNA-Fex2 DNA prepared in Example <1-1> as a template and the primers (SEQ ID NO: 11 and SEQ ID NO: 12) shown in Table 2. The PCR reaction was performed under the following conditions: 2 min at 95° C.; and 25 cycles of, 30 sec at 94° C., 30 sec at 60° C. and 30 sec at 72° C. The amplification product was digested with restriction enzymes BamHI and XhoI (TaKaRa) and inserted into the same restriction enzyme sites of a pET43.1a vector (Novagen).

TABLE 2

Primers used in preparation of
recombinant protein as antigen

| | | SEQ ID |
|---|---|---|
| Primers | Sequences | NO |
| hFex-2-4E5 | 5′-aaa aag gat cca cag tgt ttg | 11 |
| sense | ctc c-3′ | |
| hFex-2-4E5 | 5′-ttt tac tcg aga ctt ttg gga | 12 |
| antisense | gat agc-3′ | |

The above-prepared expression vector was named “pET-4E5”, and the expression and isolation of a protein were performed in the same manner as in Example <2-1>. The preparation of a monoclonal antibody was performed in Dinona Inc. (Seoul, Korea). Specifically, 20 μg of the above-prepared recombinant protein was immunized into six rats at 2-week intervals to obtain a positive hybridoma clone (clone 5G3). The hybridoma clone was injected into the abdominal cavity of rats, and the abdominal dropsy of the rats was obtained, thus obtaining the monoclonal antibody. The isotype of the monoclonal antibody was determined using the IsoStrip mouse monoclonal antibody isotyping kit (Roche). As a result, the isotype of the inventive monoclonal antibody was determined to be an IgG1 with lambda chain.

The hybridoma 5G3 that produces the inventive human FEX-2 monoclonal antibody was deposited under accession No. KCTC-10639BP on May 21, 2004 with the Korean Collection for Type Cultures (KCTC), which is an International Depository Authority under the Budapest Treaty.

### Example 3

### Determination of Expression or Non-Expression of FEX-2 on Surface of L/FEX-2 Cells

Whether or not FEX-2 is expressed on the surface of the L/FEX-2 cells prepared in Example <1-2> was analyzed by flow cytometry using the FEX-2 monoclonal antibody (5G3) prepared in Example <2-3>.

For this purpose, a confluent plates, in which L/FEX-2 cells were first cultured comprising Dulbecco's modified Eagle's medium (DMEM), after which the plate was treated with a PBS buffer solution comprising 0.25% trypsin and 0.05% EDTA so as to separate the cells from the plate surface. The cells were washed with PBS buffer solution twice and then re-suspended in PBS buffer solution. To the cell suspension, the FEX-2 monoclonal antibody (5G3) was added and cultured at 4° C. for one hour. To the reaction mixture, 10 μg/ml of FITC-conjugated secondary rabbit anti-mouse immunoglobulin G (Santa Cruz Biotechnology, Inc., CA) was added and the cells were further cultured at 4° C. for one hour. Then, the reaction mixture was analyzed at 488 nm by using a flow cytometer (Becton Dickinson, San Jose, Calif.) equipped with a 5-watt laser. As a control group, mouse immunoglobulin (IgG1) in place of the FEX-2 monoclonal antibody was used.

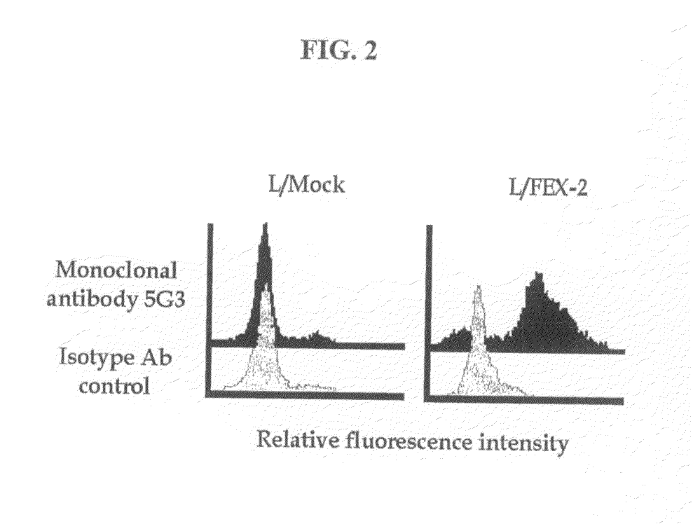

The test results showed that FEX-2 was expressed on the surface of the L/FEX-2 cells. However, FEX-2 was not expressed on the surface of the L/Mock cells. Also, even in the use of the mouse immunoglobulin in place of the FEX-2 monoclonal antibody, the expression of FEX-2 was not detected (see FIG. 2).

### Example 4

### Binding and Phagocytosis of Aged Red Blood Cells by FEX-2

<4-1> Binding and Phagocytosis of Aged Red Blood Cells by L/FEX-2 Cells

Whether or not aged red blood cells are bound and phagocytosed by the L/FEX-2 cells prepared in Example <1-2> and L/Mock cells as a control group was examined. For this purpose, normal red blood cells obtained from normal adult volunteers were first diluted with PBS to a hematocrit concentration of 20% and cultured at 37° C. for 4 days, thus preparing aged red blood cells. Then, each of L/FEX-2 cells, L/Mock cells and normal L cells was cultured to a confluent 6-well plate comprising Dulbecco's modified Eagle's medium (DMEM). To the culture media, aged red blood cells and normal red blood cells were added to a hematocrit concentration of 1% and cultured at 37° C. for one hour. Then, the cells were washed five times with PBS, and observed for the binding of the aged red blood cells and normal red blood cells using an optical microscope. The number of red blood cells bound to 100 randomly selected L/FEX-2 cells was counted and averaged. To analyze phagocytosis, the cells were washed with PBS and treated with water for 10 seconds to completely dissolve undigested cells. Then, the L/FEX-2 cells were stained using the Diff quick staining kit (IMEM Ins., San Marcos, Calif., USA), and five sites from the stained cells were randomly selected and counted for the number of cells which have phagocytosed red blood cells phagocytosed. Binding and phagocytosis indexes (%) were expressed as the percentages of cells having red blood cells bound and phagocytosed thereby, relative to the entire L/FEX-2 cells.

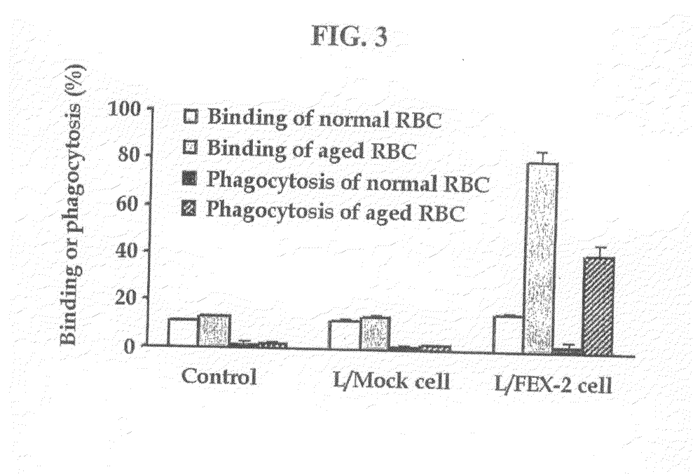

The test results showed that the aged red blood cells were bound and phagocytosed by the L/FEX-2 cells. However, the aged red blood cells were little bound and phagocytosed by the normal L cells and L/Mock cells as control groups. Also, the normal red blood cells were little bound and phagocytosed by the L/FEX-2 cells (see FIG. 3).

These results suggest that FEX-2 mediates the selective binding and phagocytosis of aged cells.

<4-2> Inhibition of Binding and Phagocytosis of Aged Red Blood Cells by Anti-FEX-2 Monoclonal Antibody.

In order to confirm if the binding and phagocytosis of aged red blood cells is attributable to FEX-2, an examination was made to check whether or not the monoclonal antibody (5G3) to FEX-2 inhibits the binding and phagocytosis of aged red blood cells.

To the L/FEX-2 cells prepared in Example <1-2>, 100 μg/ml of the human FEX-2 monoclonal antibody (5G3) was added and the mixture was pre-cultured at 37° C. for one hour. As a control group, mouse immunoglobulin (IgG1) in place of the FEX-2 monoclonal antibody was used. To the culture medium, the aged red blood cells prepared in Example <4-1> were added to a hematocrit concentration of 1%, and the cell mixture was additionally cultured at 37° C. for one hour. After completion of the culture, the binding and phagocytosis of the aged red blood cells were analyzed in the same manner as in Example <4-1>. In this regard, the binding and phagocytosis of the aged red blood cells were expressed as inhibition percentage relative to the case of L/FEX-2 cells, which have not been pre-cultured with the antibody.

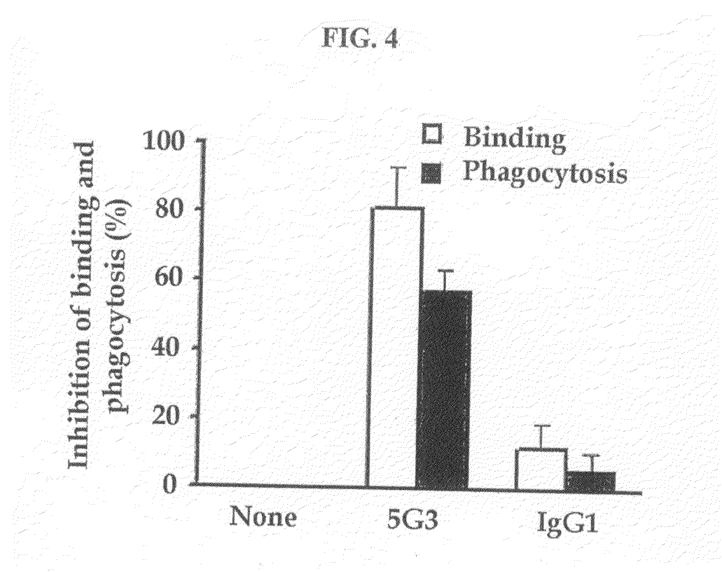

The test results showed that the pre-culture of the L/FEX-2 cells with the FEX-2 monoclonal antibody (5G3) resulted in the significant inhibition of the binding and phagocytosis of aged red blood cells. On the other hand, the pre-culture of the L/FEX-2 cells with the mouse immunoglobulin had no significant effect on the binding and phagocytosis of aged red blood cells (see FIG. 4).

The above test results suggest that the binding and phagocytosis of aged red blood cells are mediated by FEX-2 and inhibited by the anti-FEX-2 antibody.

### Example 5

### Identification of Recognition Sites of FEX-2 in Binding of Aged Red Blood Cells to FEX-2

<5-1> Measurement of Expression of Phosphatidylserine on Cell Surface According to the Aging of Red Blood Cells

Apoptotic cells exhibit: an “eat-me” signal on the cell surface so as to be phagocytosed by macrophages. A typical example of the signal is phosphatidylserine. Thus, it was assumed that, in the binding and phagocytosis of aged red blood cells by FEX-2, phosphatidylserine expressed on the surface of aged red blood cells would be specifically recognized by FEX-2. To confirm this assumption, the expression of phosphatidylserine according to the degree of aging was first measured. Annexin V is a protein which binds to phospholipid in a calcium-dependent manner and is known to have a strong affinity for phosphatidylserine. In view of this fact, the amount of phosphatidylserine expressed on the cell surface during the aging of red blood cells was measured using the annexin V antibody by flow cytometry. Namely, normal red blood cells obtained from normal adult volunteers were cultured in PBS at 37° C. for each of 0, 2 and 4 days, and then, the cells were measured for the expression of phosphatidylserine by flow cytometry using the annexin V apoptosis detection kit according to the manufacturer's instruction.

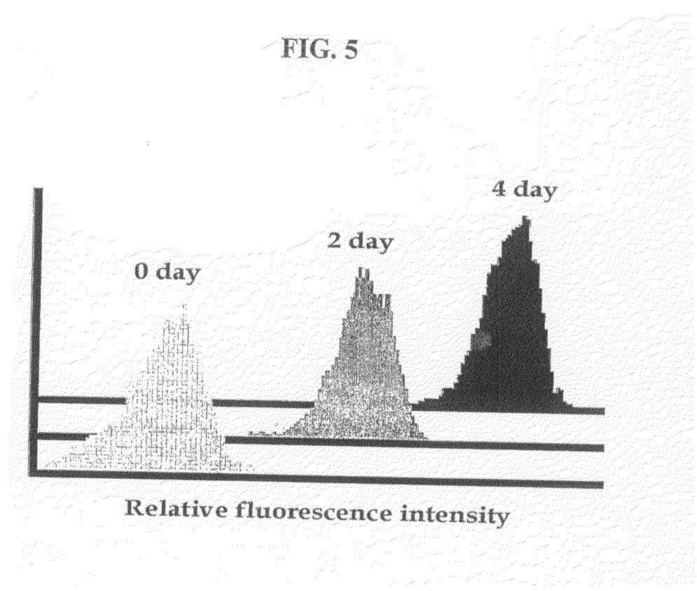

The test results showed that the expression of phosphatidylserine on the surface of red blood cells was increased with the progression of aging (see FIG. 5).

<5-2> Examination of Binding of Red Blood Cells by FEX-2 According to Aging of Red Blood Cells

The L/FEX-2 cells were cultured with aged red blood cells in the same manner as in Example <4-1>. In this regard, the aged red blood cells were used after culture for each of 0, 1, 2, 3, 4 and 5 days. After completion of the culture, the binding of the aged red blood cells to the L/FEX-2 cells was analyzed in the same manner as in Example <4-1>.

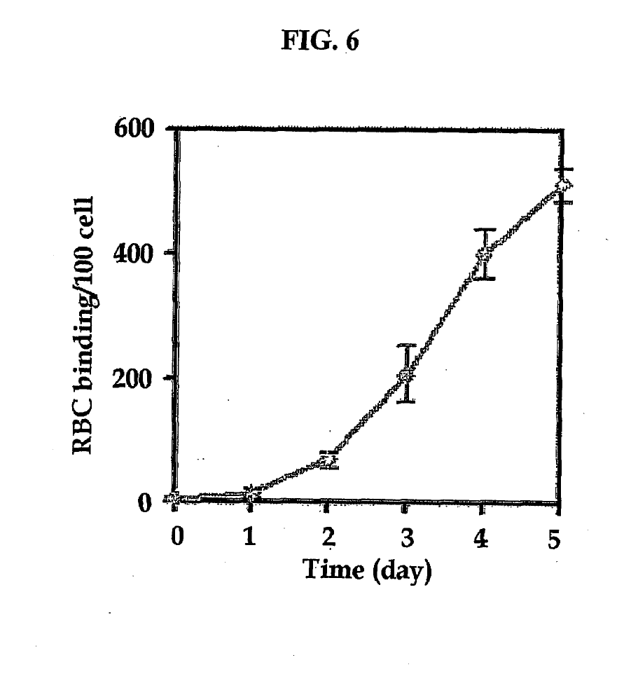

The test results showed that, as the culture time of the red blood cells became longer (i.e., the aging of the red blood cells progressed), the binding of the red blood cells to the L/FEX-2 cells increased (see FIG. 6).

<5-3> Activity of Binding of L/FEX-2 Cells to Various Phospholipids

In order to confirm the fact that phosphatidylserine on the cell surface is increased with the progression of aging and recognized by FEX-2, the activity of the L/FEX-2 binding to various phospholipids was examined. Liposomes comprising phospholipids, such as phosphatidylcholine (PC), phosphatidylserine (PS), phospatidylinositol (PI), phsophatidylethanolamine (PE), phosphatidic acid (PA) and phosphatidylgycerol (PG), were prepared using a previously reported method (Oka, K. et al., Proc. Natl. Acad. Sci. USA. 95:9535-9540, 1998; Fadok, V. A. et al., Nature 405:85-90, 2000). Specifically, PS:PC (PS liposome), PI:PC (PI liposome), PG:PC (PG liposome), PE:PC (PE liposome), and PA:PC (PA liposome), were mixed with each other at a molar ratio of 50:50 to prepare liposomes, and a liposome comprising only PC (PC liposome) was prepared. Each of the liposome mixtures was mixed with chloroform and dried under nitrogen gas. Then, each of the mixtures was re-suspended in PBS to a final concentration of 5 mM and sonicated on ice for 10 minutes. Also, during the preparation of the liposomes, N-(lissamin rhodamine B sulfonyl)-L-α-phosphatidylethanolamine (Avanti Polar Lipids) was added at a concentration of 1% relative to the total phospholipid so as to fluorescence-labeled phospholipid liposomes. The L/FEX-2 cells prepared in Example <1-2> were cultured to confluence in DMEM medium, to which 100 μM of each of the above-prepared phospholipid liposomes was added and followed by culturing at 37° C. for 1 hour. After completion of the culture, the cells were sufficiently washed with PBS and analyzed for the binding of liposomes to the L/FEX-2 cells in the same manner as in Example <4-1>. Also, to measure the phagocytosis of the phospholipid liposomes, the L/FEX-2 cells were added and dissolved in cell lysis buffer (1% Triton X-100). Then, the amount of phagocytosed liposomes was measured with the fluorescent microplate reader (Biolumin 960, Molecular Dynamics). In this regard, the excitation wavelength was 549 nm, and the emission wavelength was 565 nm. As a control group, L/Mock cells were used.

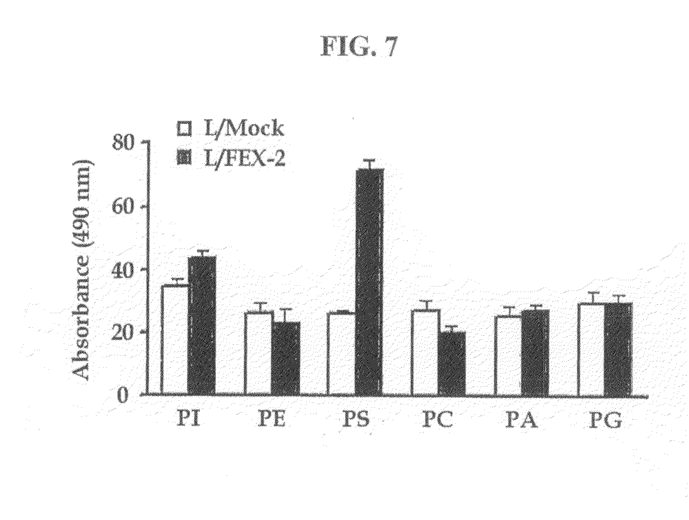

The test results showed that the binding of the phospholipid liposomes to the L/FEX-2 cells was the highest for the PS-liposome, and similar to the control group for the other phospholipid liposomes (see FIG. 7). Also, the phagocytosis of the phospholipid liposomes by the L/FEX-2 cells was much higher for the PS liposome than those of the other phospholipid liposomes and the control group (data not shown).

<5-4> Inhibitory Effects of Various Phospholipid Liposomes on Binding and Phagocytosis of Aged Red Blood Cells by L/FEX-2 Cells

Whether the binding and phagocytosis of red blood cells by the L/FEX-2 cells are inhibited, it was examined by pre-culturing the L/FEX-2 cells with each of the PS liposome, PI liposome, PG liposome, PE liposome, PA liposome and PC liposome prepared in Example <5-3> and then additionally culturing the pre-cultured cells with aged red blood cells. Specifically, the L/FEX-2 cells were cultured in the same manner as in Example 4-1, in which each 100 μM of the PS liposome, the PI liposome, the PG liposome, the PE liposome, the PA liposome and the PC liposome was added and cultured at 37° C. for 30 minutes. To a control group, the phospholipid liposome was not added. The L/FEX-2 cells were treated and cultured with aged red blood cells in the same manner as in Example <4-2>. After completion of the culture, the binding and phagocytosis of the aged red blood cells were analyzed in the same manner as in. Example <4-1>. The binding and phagocytosis of aged red blood cells were expressed as values relative to the control group taken as 100%.

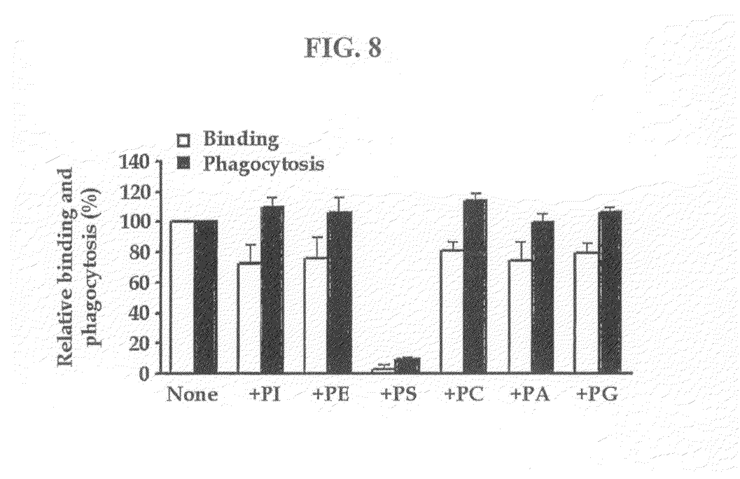

The test results showed that, in the case where the L/FEX-2 cells were pre-cultured with the PS liposome, the binding and phagocytosis of aged red blood cells were highly inhibited. On the other hand, in the case where the L/FEX-2 cells were pre-cultured with each of the liposomes other than the PS liposome, the binding of aged red blood cells was not substantially inhibited and the phagocytosis of aged red blood cells was only slightly reduced (see FIG. 8).

<5-5> Inhibition of Binding of Aged Red Blood Cells to L/FEX-2 Cells According to Addition Concentration of PS Liposome

The binding and phagocytosis of red blood cells were examined by pre-culturing the L/FEX-2 cells with varying concentrations of the PS liposome prepared in Example <5-3> and then culturing the pre-cultured cells with aged red blood cells. To a control group, the PC liposome in place of the PS liposome was added. Specifically, the test was performed in the same manner as in Example <5-4> except that the PS liposome was added at each of concentrations of 0, 0.1, 1, 10 and 100 μM. After completion of the culture, the binding of the aged red blood cells to the L/FEX-2 cells was measured in the same manner as in Example <4-1>.

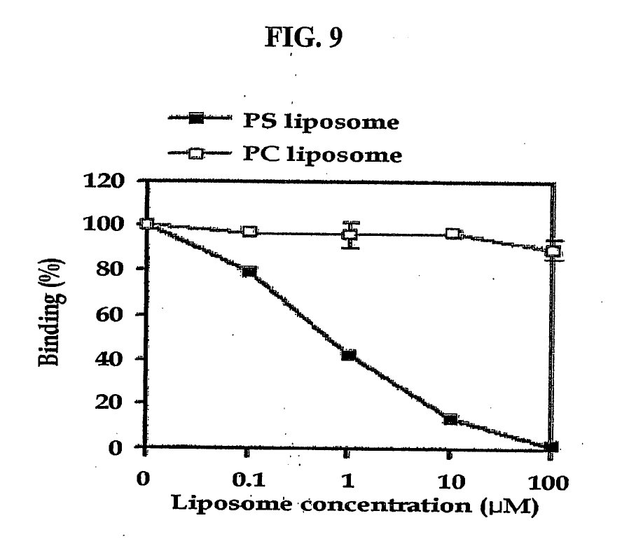

The test results showed that the pre-culture of the L/FEX-2 cells with the PS liposome resulted in the inhibition of the binding of the aged red blood cells to the L/FEX-2 cells in a manner dependent on the addition concentration of the PS liposome. On the other hand, the pre-culture of the L/FEX-2 cells with the PC liposome had no effect on the binding of the aged blood cells to the L/FEX-2 cells (see FIG. 9).

The test results suggest that FEX-2 specifically recognizes phosphatidylserine expressed on the surface of aged red blood cells so as to bind and phagocytize the aged cells.

<5-6> Inhibitory Effect of Structural Analogues of Phosphatidylserine on Binding and Phagocytosis of Aged Red Blood Cells by L/FEX-2 Cells

In order to further confirm whether FEX-2 specifically recognizes the PS liposome, phospho-L-serine (PLS) and phospho-D-serine (PDS), which are structural analogue of phosphatidylserine, were used to examine the inhibition of the binding and phagocytosis of aged red blood cells by the L/FEX-2 cells.

The test was performed in the same manner as in Example <5-4> except that each of PS liposome, phospho-L-serine and phospho-D-serine (Sigma) was used as the phospholipid liposome. To a control group, the phospholipid liposome was not added. The binding and phagocytosis of the aged red blood cells were expressed as values relative to the control group taken as 100%.

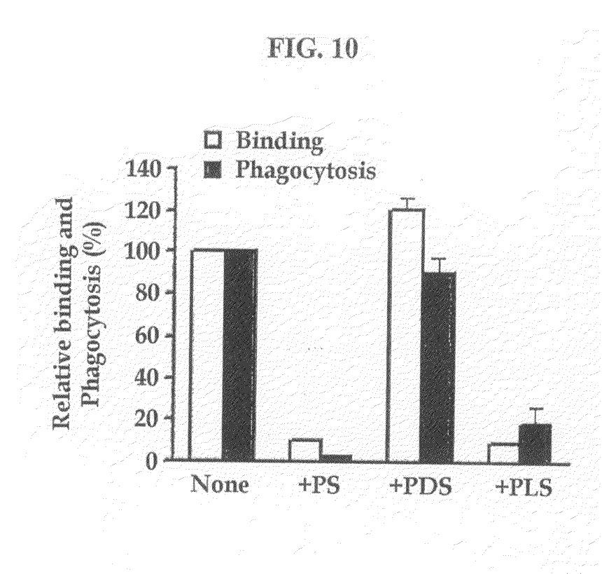

The test results showed that the pre-culture of the L/FEX-2 cells with the PDS had no significant effect on the binding and phagocytosis of the aged red blood cells. On the other hand, the pre-culture of the L/FEX-2 cells with the PLS liposome resulted in the significant inhibition of the binding and phagocytosis of the aged red blood cells, which was similar to the case of PS liposome. (see FIG. 10)

These test results suggest that FEX-2 does not recognize phosphatidylserine through negative charges, but rather specifically recognizes the structure of phosphatidylserine.

### Example 6

### Binding and Phagocytosis of Apoptotic Cells by FEX-2

<6-1> Induction of Cell Apoptosis

The expression of phosphatidylerine on the cell surface is widely known as an “eat-me” signal not only for aged cells, but also for apoptotic cells. On the basis of this fact, in order to confirm whether FEX-2 also mediates the binding and phagocytosis of apoptotic cells, the apoptosis of cells was induced to prepare apoptotic cells.

For this purpose, the apoptosis of human leukemic Jurkat T cells (Korean cell line bank) and premyelomonocytic leukemic U937 cells (Korean cell line bank) was induced. The cells were cultured in an RPMI 1640 medium comprising 10% fetal bovine serum. The induction of apoptosis of the Jurkat T cells was performed according to a known method by inoculating the Jurkat T cells in a medium comprising 100 ng/ml of an anti-Fas antibody (CH-11) and culturing the inoculated cells for 6 hours (Oka, K. et al., Proc Natl Acad Sci USA, 95:9535-40, 1998). Also, the induction of apoptosis of the U937 cells was performed according to a known method by inoculating the U937 cells in a medium comprising 100 ng/ml of ectoposide and culturing the inoculated cells for 4 hours (Bave, U., et. al., J Immunol 165:3519-26, 2000). The degree of apoptosis of the cells was determined by measuring the amount of phosphatidylserine expressed on the cell surface, by flow cytometry in the same manner as in Example <5-1> using the annexin V apoptosis detection kit (Santacruz) according to the manufacturer's instruction.

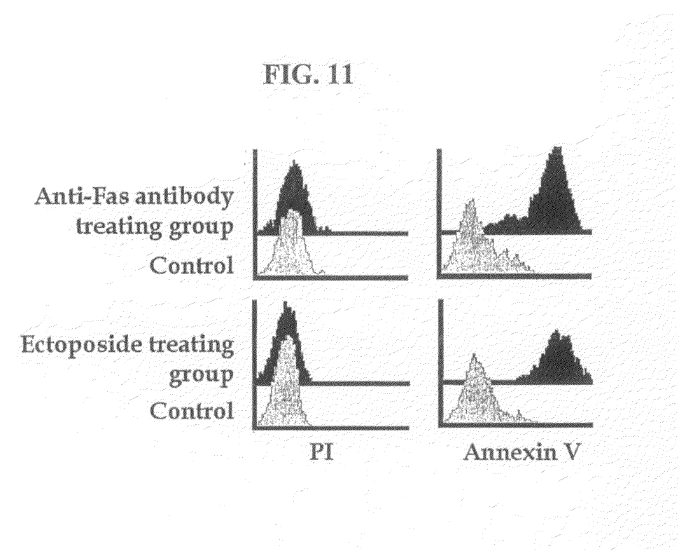

The test results showed that the apoptosis of the cells was induced in both the Jurkat T cells treated with the anti-Fas antibody and the U937 cells treated with ectoposide (see FIG. 11).

<6-2> Phagocytosis of Apoptotic Cells by L/FEX-2 Cells

The apoptosis-induced Jurkat T cells and U937 cells prepared in Example <6-1> were cultured with the L/FEX-2 cells prepared in Example <1-2> and then analyzed for phagocytosis. Specifically, the L/FEX-2 cells were cultured to confluence in a 6-well plate, after which 1×10⁶ of the apoptotic Jurkat T cells or U937 cells were added thereto and cultured at 37° C. for one hour. After completion of the culture, the cells were washed five times with PBS, and stained in the same manner as in Example <4-1> using the Diff quick staining kit (IMEM Ins., San Marcos, Calif., USA). Then, five sites of the stained cells were randomly selected and counted for the number of cells which have phagocytized the apoptotic cells.

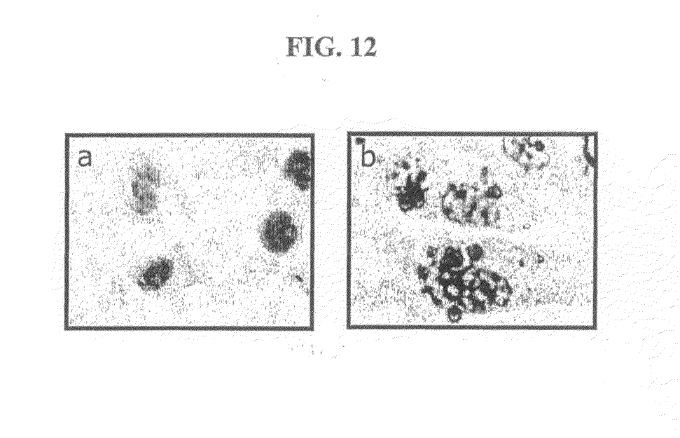

The test results showed that, in the control group L/Mock cells, the apoptosis-induced Jurkat T cells were little phagocytized (see FIG. 12a), but in the L/FEX-2 cells, a large number of the apoptotic Jurkat T cells were phagocytized (see FIG. 12b). Also, the test using the U937 cells provided results similar to the above results (data not shown). The L/FEX-2 cells did not phagocytize the Jurkat T cells and U937 cells whose apoptosis had not been induced.

<6-3> Inhibitory Effects of PS Liposome and FEX-2 Monoclonal Antibody on Phagocytosis of Apoptotic Cells

In order to confirm if the phagocytosis of apoptotic cells by the L/FEX-2 cell in Example <6-2> occurs because FEX-2 recognizes phosphatidylserine expressed on the surface of apoptotic cells, whether the PS liposome and the FEX-2 monoclonal antibody inhibit the phagocytosis of apoptotic cells by the L/FEX-2 cells was examined.

For this purpose, the L/FEX-2 cells prepared in Example <1-2> were cultured to confluence in a 6-well plate comprising DMEM medium. Then, the cells were pre-cultured with 100 μg/ml of the FEX-2 monoclonal antibody (5G3) prepared in Example <2-3>, 100 μg/ml of mouse immunoglobulin (IgG1) and 100 μM of the PS liposome and PC liposome prepared in Example <6-3>. To the culture media, 1×10⁶ of the apoptosis-induced Jurkat T cells or U937 cells prepared in Example <6-1> were added, and the cell mixture was additionally cultured at 37° C. for one hour. After completion of the culture, the cells were washed five times with PBS and stained in the same manner as in Example <4-> using the Diff quick staining kit (IMEM Ins., San Marcos, Calif., USA). Then, five sites from the stained cells were randomly selected and counted for the number of cells which have phagocytized the apoptotic cells. The counted number of the L/FEX-2 cells which phagocytosed apoptotic cells was expressed as inhibition percentage of phagocytosis relative to the case where the L/FEX-2 cells were cultured only with apoptotic cells.

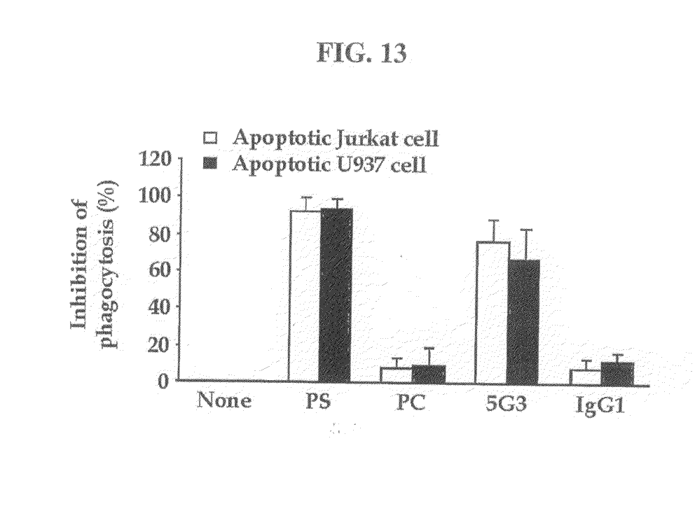

The test results showed that the phagocytosis of apoptotic cells by the L/FEX-2 cells was almost completely inhibited by phosphatidylserine and the FEX-2 monoclonal antibody. On the other hand, the inhibition of phagocytosis by the PC liposome and mouse immunoglobulin was insignificant (see FIG. 13).

These results suggest that FEX-2 recognizes phosphatidylserine expressed on the surface of not only aged cells but also apoptotic cells and mediates the phagocytosis of them.

<6-4> Analysis of Anti-Inflammatory Cytokine Secreted from L/FEX-2 Cells by Treatment with FEX-2 Monoclonal Antibody

The L/FEX-2 cells prepared in Example <1-2> were cultured to confluence in a 6-well plate comprising DMEM medium, and then, each of 100 μg/ml of the FEX-2 monoclonal antibody (5G3) prepared in Example <2-3> and 100 μg/ml of mouse immunoglobulin was added thereto, and the cells were additionally cultured. After completion of the culture, the culture media were recovered and measured for the production of TGF-β by ELISA (enzyme-linked immunosorbent assay).

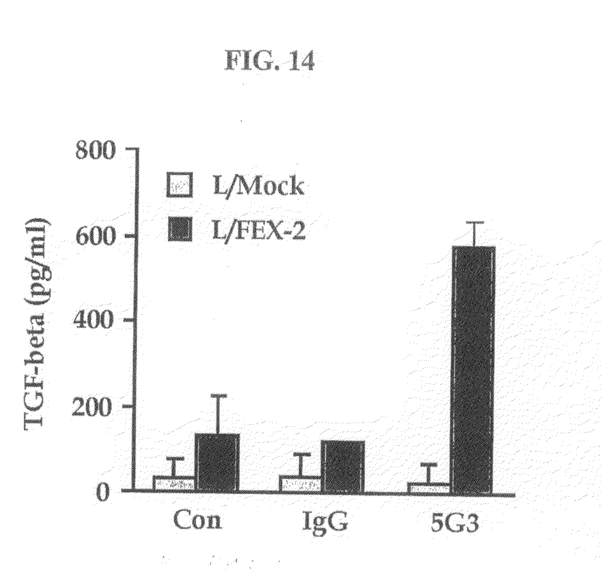

The test results showed that the addition of the inventive FEX-2 monoclonal antibody stimulated the production of anti-inflammatory cytokine TGF-β (see FIG. 14).

### Example 7

### Phagocytosis of Apoptotic Cells by FEX-2 Expressed on Surface of Macrophages

<7-1> Examination of Expression or Non-Expression of FEX-2 on Macrophages

Whether FEX-2 is expressed on macrophages involved in the phagocytosis of apoptotic cells and aged cells was examined. For this purpose, blood from healthy adult volunteers was first obtained, and monocytes isolated from the blood were cultured in an X-Vivo 10 medium (BioWhitaker) comprising 10% human serum for about 7 days so as to differentiate into macrophages. The macrophages were activated by treatment with β-glucan, thus preparing human monocyte-derived macrophages (HMDM). Meanwhile, the THP-1 human monocyte cell line (Korean cell line bank) was treated with PMA to a concentration of 10 ng/ml and cultured for 72 hours so as to differentiate into macrophages. To examine if FEX-2 is expressed on the surface of the above two kinds of macrophages and the mouse macrophage cell line P388D1 (Korean cell line bank), flow cytometry analysis was carried out using the FEX-2 monoclonal antibody 5G3 prepared in Example 2.

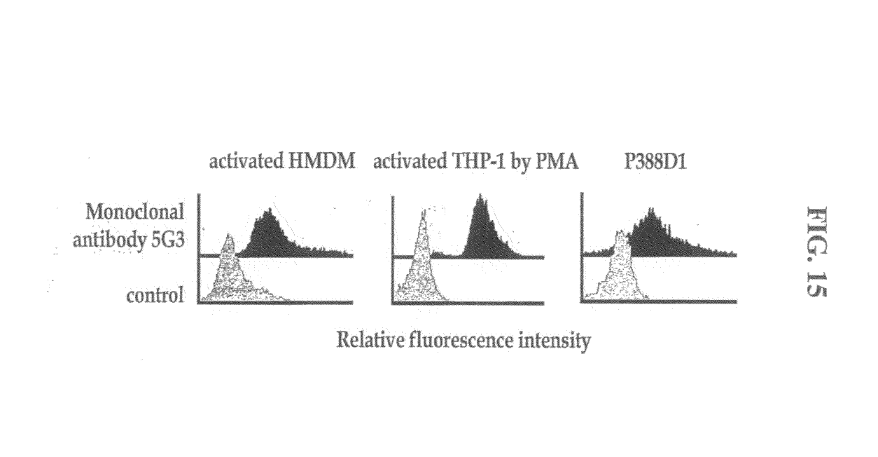

The test results showed that FEX-2 was expressed in all the HMDM, THP-1 and P388D1 macrophages (see FIG. 15).

<7-2> Inhibitory Effects of PS Liposome and FEX-2 Monoclonal Antibody on Phagocytosis of Apoptotic Cells by Macrophages

To examine if the phagocytosis of apoptotic cells by the above three kinds of macrophages prepared in Example <7-1> is inhibited by the PS liposome and the FEX-2 monoclonal antibody, the following test was carried out.

The above three kinds of macrophages prepared in Example <7-1> were first pre-cultured for 30 minutes with the PS liposome prepared in Example <5-3> or the FEX-2 monoclonal antibody prepared in Example <2-3>. To the culture media, 1×10⁶ of the apoptosis-induced Jurkat T cells or U937 cells prepared in Example <6-1> were added, and the cell mixture was additionally cultured at 37° C. for one hour.

As a control group, mouse immunoglobulin (IgG1) in place of the FEX-2 monoclonal antibody was used. The inhibition of phagocytosis of the macrophages was measured in the same manner as in Example <6-3>.

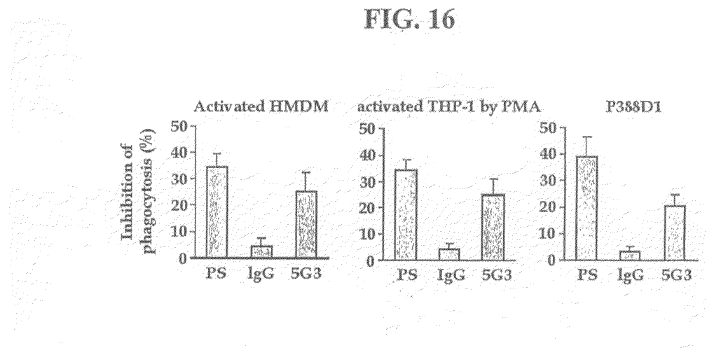

The test results showed that the phagocytosis by all the activated HMDM, THP-1 and P388D1 macrophages was inhibited by the PS liposome and the FEX-2 monoclonal antibody 5G3. On the other hand, it was not substantially inhibited by the mouse immunoglobulin (see FIG. 16).

The test results suggest that PEX-2 expressed on the surface of macrophages specifically recognizes phosphatidylserine of apoptotic cells to mediate binding and phagocytosis of apoptotic cells by macrophages.

<7-3> Analysis of Anti-Inflammatory Cytokine Secreted from Macrophages by Treatment with FEX-2 Ligand

It is known that the phosphatidylserine-dependent phagocytosis of apoptotic cells stimulates the secretion of anti-inflammatory cytokine TGF-β and inhibits the secretion of inflammatory cytokine TNF-α (Fadok V. A. et al., J. Clin. Invest., 101:890-898, 1998; McDonald P. P. et al., J. Immunol. 163:6164-6172, 2000). In view of this fact, activated macrophages were treated with each of various ligands specific to FEX-2 expressed on macrophages, and the secretion of anti-inflammatory cytokine or inflammatory cytokine from the macrophages by treatment with the ligands was measured. For this purpose, the mouse macrophage cell line J774 (Korean cell line bank) was first stimulated by treatment with LPS (10 ng/ml), after which 100 μM of the PS liposome or the PC liposome, aged red blood cells, 100 μg/ml of each of the FEX-2 monoclonal antibody 5G3 and the mouse immunoglobulin IgG were added thereto. The mixture was cultured at 37° C. for 18 hours. After completion of the culture, the culture media were recovered and were measured for the amounts of TGF-β and TNF-α using ELISA (enzyme-linked immunosorbent assay). A control group was untreated.

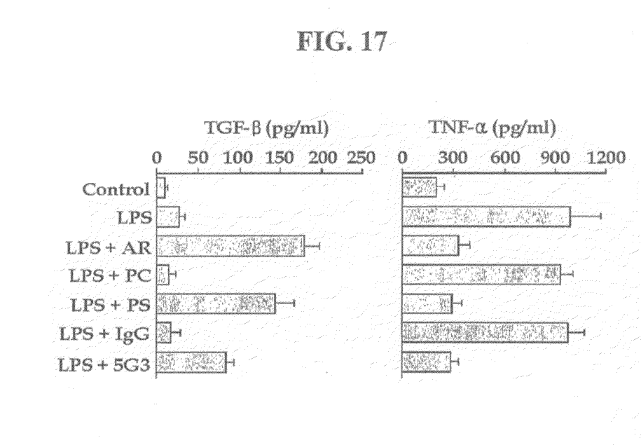

The test results showed that, in the macrophages stimulated by treatment with LPS alone, the secretion of inflammatory cytokine TNF-α increased. Meanwhile, in the case where the macrophages were treated with LPS and then the aged red blood cells, the PS liposome and the FEX-2 monoclonal antibody 5G3, the secretion of anti-inflammatory cytokine TGF-β in the macrophages was promoted and the secretion of inflammatory cytokine TNF-α was inhibited. On the other hand, the macrophages treated with the PC liposome and the mouse immunoglobulin IgG showed essentially the same aspect as the macrophages stimulated with LPS (see FIG. 17).

These results suggest that the PS liposome and FEX-2 monoclonal antibody acting as ligands specific to the FEX-2 protein expressed on the surface of macrophages allow the same signal to be transferred as in the case of the phagocytosis of aged cells by macrophages and thus stimulates the secretion of anti-inflammatory cytokine from macrophages, thereby inhibiting inflammation.

### INDUSTRIAL APPLICABILITY

As can be seen from the foregoing, the inventive ligand, specific to the FEX-2 polypeptide, can bind to FEX-2 expressed on the surface of phagocytes so that it can inhibit the secretion of inflammatory cytokines from the phagocytes and stimulate the secretion of anti-inflammatory cytokines, so as to treat or prevent inflammatory diseases.

1-30. (canceled)

31. A method for modulating the secretion of an inflammation-associated cytokine in a subject, the method comprising administering to a subject in need thereof an effective amount of a FEX-2 polypeptide specific ligand except phosphatidylserine.

32. The method of claim 31, wherein the cytokine is secreted from phagocytes.

33. The method of claim 31, wherein modulating the secretion of the cytokine is stimulating the secretion of anti-inflammatory cytokine and/or inhibiting the secretion of inflammatory cytokine.

34. The method of claim 33, wherein the anti-inflammatory cytokine is selected from the group consisting of TGF-β, IL-10, IL-4 and IL-13.

35. The method of claim 33, wherein the inflammatory cytokine is selected from the group consisting of TNF-α, IL-1, IL-6, IL-8, IL-18 and MIP₂.

36. The method of claim 31, wherein the FEX-2 polypeptide is derived from a mammal.

37. The method of claim 31, wherein the FEX-2 polypeptide comprises the amino acid sequence set forth in SEQ ID NO. 1.

38. The method of claim 31, wherein the FEX-2 polypeptide is expressed on the surface of phagocytes.

39. The method of claim 31, wherein the ligand is selected from the group consisting of phosphatidylserine derivatives and anti-FEX-2 antibody.

40. The method of claim 39, wherein the phosphatidylserine derivative is phospho-L-serine.

41. The method of claim 39, wherein the anti-FEX-2 antibody is a polyclonal or monoclonal antibody.

42. The method of claim 41, wherein the monoclonal antibody is produced by a hybridoma (accession number: KCTC 10639BP).

43. A method for treating or preventing an inflammatory disease, the method comprising administering to a subject in need thereof an effective amount of a FEX-2 polypeptide specific ligand except phosphatidylserine.

44. The method of claim 43, wherein the FEX-polypeptide is derived from a mammal.

45. The method of claim 43, wherein the FEX-2 polypeptide comprises the amino acid sequence set forth in SEQ ID NO: 1.

46. The method of claim 43, wherein the FEX-2 polypeptide is expressed on the surface of phagocytes.

47. The method of claim 43, wherein the ligand is selected from the group consisting of phosphatidylserine derivatives and anti-FEX-2 antibody.

48. The method of claim 47, wherein the phosphatidylserine derivative is phospho-L-serine.

49. The method of claim 47, wherein the anti-FEX-2 antibody is a polyclonal or monoclonal antibody.

50. The method of claim 49, wherein the monoclonal antibody is produced by the hybridoma (accession number: KCTC 10639BP).

51. The method of claim 43, wherein the inflammatory disease is selected from the group consisting of inflammation, inflammatory bowl disease, diabetic ocular disease, peritonitis, osteomyelitis, cellulitis, meningitis, encephalitis, pancreatitis, trauma causing shock, bronchial asthma, rhinitis, sinusitis, otitis media, pneumonia, gastritis, enteritis, cystic fibrosis, apoplexy, bronchitis, bronchiolitis, hepatitis, nephritis, arthritis, gout, spondylitis, Reiter's syndrome, polyarteritis nodosa, hypersensitivity vasculitis, Wegener's granulomatosis, polymyalgia rheumatica, giant cell arteritis, calcium crystal deposition arthropathy, pseudogout, nonarticular rheumatism, bursitis, tenosynovitis, epicondylitis (Tennis elbow), neuropathic joint disease (Charcot's joint), hemarthrosis, Henoch-Schonlein Purpura, hypertrophic osteoarthropathy, multicentric reticulohistiocytoma, scoliosis, hemochromoatosis, sickle cell disease and other hemoglobinopathies, hyperlipoproteinemia, hypogammaglobulinemia, hyperparathyroidism, acromegaly, familial mediterranean fever, Behcet's disease, systemic lupus erythematosus, relapsing fever, psoriasis, multiple sclerosis, septicemia, septic shock, acute respiratory distress syndrome, multiple organ failure, chronic obstructive pulmonary disease, acute lung injury and broncho-pulmonary dysplasia.

52. A method for screening an agent for modulating the secretion of an inflammation-associated cytokine, the method comprising the steps of:
(a) culturing a phagocyte expressing FEX-2 with a test agent; and
(b) measuring change in the level of cytokine secreted from the phagocyte cultured with the test agent relative to the level of cytokine secreted from the phagocyte cultured without the test agent.

53. The method of claim 52, wherein the cytokine secreted from the phagocyte in the step (b) is TGF-β or TNF-α.

54. A method for identifying an agent for treating or preventing an inflammatory disease, the method comprising the steps of:
(a) culturing a phagocyte expressing FEX-2 with a test agent;
(b) measuring change in the level of cytokine secreted from the phagocyte cultured with the test agent relative to the level of cytokine secreted from phagocyte cultured without the test agent;
(c) administering the test agent selected in the step (b), which modulates the level of the cytokine selected from the phagocyte, to an animal suffering from an inflammatory disease, and determining whether the test agent shows a therapeutic effect in the animal.

55. The method of claim 54, wherein the cytokine secreted from the phagocyte in the step (b) is TGF-β or TNF-α.

56. The method of claim 54, wherein the test agent used in the step (c) has the activity to stimulate the secretion of anti-inflammatory cytokine and/or inhibit the secretion of inflammatory cytokine.

57. Use of a FEX-2 polypeptide specific ligand except phosphatidylserine, for preparing a pharmaceutical composition for modulating the secretion of an inflammation-associated cytokine.

58. Use of a FEX-2 polypeptide specific ligand except phosphatidylserine, for preparing a pharmaceutical composition for treating or preventing an inflammatory disease.

59. A pharmaceutical composition for modulating the secretion of an inflammation-associated cytokine, the composition comprising a FEX-2 polypeptide specific ligand except phosphatidylserine, as an active ingredient.

60. A pharmaceutical composition for treating or preventing an inflammatory disease, the composition comprising a FEX-2 polypeptide specific ligand except phosphatidylserine, as an active ingredient.
